(19) United States
(12) Patent Application Publication
(10) Pub. No.: US 2016/0298187 A1
(43) Pub. Date: Oct. 13, 2016

(54) METHODS OF TAGGING PARTICLES FOR MULTIPLEXED FUNCTIONAL SCREENING

(71) Applicant: Verily Life Sciences LLC, Mountain View, CA (US)

(72) Inventors: Jerrod Joseph SCHWARTZ, San Francisco, CA (US); Vikram Singh BAJAJ, Mountain View, CA (US); Alberto Clemente VITARI, San Francisco, CA (US)

(21) Appl. No.: 14/681,601

(22) Filed: Apr. 8, 2015

(51) Int. Cl. C12Q 1/68; C12N 15/11

(52) U.S. Cl. CPC C12Q 1/6876; C12N 15/11; C12Q 2600/158

(57) ABSTRACT

The present invention relates generally to the fields of cell biology and laboratory diagnostics, and particularly to general compositions and of uniquely tagged particles linked to moieties of known properties and methods of making tagged, functionalized particles. Additionally, the invention relates to methods of screening a collection of tagged functionalized particles.

The sequence listing submitted herewith is incorporated by reference in its entirety.

### FIELD OF THE DISCLOSURE

The present disclosure relates generally to the fields of cell biology and laboratory diagnostics and particularly to compositions of uniquely tagged, functional molecules and general methods to uniquely tag a multiplicity of molecules to facilitate identifying those having desired biological activity.

### BACKGROUND OF THE DISCLOSURE

In the search for molecules of interest for in vivo targeting, drug delivery, therapeutic use, and monitoring of specific analytes, there is a vast combinatorial space of properties that can be explored. Size, shape, charge, magnetic and optical properties, composition, surface modification, and targeting groups are some of the characteristics that can be varied, each of which will result in different formulation, delivery, pharmacokinetic, targeting, and detection properties. The huge number of different possible combinations makes it virtually impossible to examine all combinations on their own using a serial approach. Methods are needed to increase the rate at which such combinations can be examined.

### SUMMARY OF THE DISCLOSURE

It is against the above background that the present disclosure provides certain advantages and advancements over the prior art. The present disclosure relates to general methods of tagging a functionalized particle with a tag that uniquely identifies each type of functionalized particle. A plurality of different tagged particles can then be pooled, and this complex mixture of tagged functionalized particles can then be introduced into an in vitro or in vivo assay to select for specific properties. Particles that demonstrate the desired property can then be isolated (e.g., by virtue of their location or concentration in the body, binding affinity, chemical properties, etc.) and their tags can be read. This facilitates easy identification of functionalized particles having the desired property.

As described more fully below, a “tag” is a chemical moiety that uniquely identifies the particle to which it is linked, a “functional moiety” is a chemical or biological moiety to be tested for a desired property, a “particle” is a chemical or physical moiety that serves as a substrate or carrier for one or more functional moieties to which the tag can also be linked, and a “functionalized particle” is a particle to which a functional moiety is associated (e.g., covalently or otherwise linked).

In one aspect, the disclosure provides a tagged, functionalized particle comprising one or more functional moieties and a unique tag linked to a particle.

In another aspect, the disclosure provides methods of preparing a tagged, functionalized particle comprising linking one or more functional moieties and a unique tag to a particle.

In certain embodiments, the tag is a nucleic acid, a peptide, an optically-encoded tag, or a mass-encoded tag. In certain embodiments, the functional moiety is a monoclonal antibody, a polyclonal antibody, single chain antibody, a single domain antibody or nanobody, a bi-specific antibody, an affibody molecule, a peptide, a peptoid, an aptamer or other nucleic acid or a small molecule or other chemical compound. In some embodiments the particle is a polymer matrix. In some embodiments, a collection of functionalized particles is prepared by pooling two or more types of functionalized particle, each type of functionalized particle comprising a unique combination of tag and functional moiety or moieties.

In yet another aspect, the disclosure provides a method of screening a collection of uniquely tagged, functionalized particles to identify a functional moiety or combination of functional moieties having a desired property or properties, the method comprising introducing the collection of tagged, functionalized particles into an assay to select for a specific property or properties; isolating the tagged, functionalized particles that manifest the desired property or properties; identifying the tags of the isolated tagged, functionalized particles; and determining the functional moiety or combination of functional moieties from the identity of the tag.

The assay can be in vitro or in vivo, and the desired property can be a location, a concentration, a binding affinity, or a chemical property of the functionalized particle or combination of functionalized particles. In some embodiments, the unique tags are identified by reading the sequences of nucleic acid tags; conducting mass spectrometry of peptide or mass-encoded tags; or conducting flow cytometry or microscopy of tags that are fluorescent dyes, quantum dots or nanodiamonds.

These and other features and advantages of the present disclosure will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

A better understanding of the disclosure may be obtained in light of the following drawings which are set forth for illustrative purposes, and should not be construed as limiting the scope of the disclosure in any way.

FIG. 1 shows a schematic for a unique nucleic acid tag.

FIG. 2 shows a schematic for amplification of unique nucleotide tags.

FIG. 3 shows a schematic of the primers used to prepare the samples for sequencing (forward primer is SEQ ID NO:01; reverse primer is SEQ ID NO:07; and target is SEQ ID NO:05).

FIG. 4 shows a graph of the particle barcodes that were counted and rank ordered as a function of abundance.

FIG. 5 shows an illustration a method of combining single cell profiling with tagged, functionalized particles of tumor cells. The heterogeneous population of cells is then exposed to a collection of uniquely tagged functionalized particles where each particle is linked to a functional moiety or combination of moieties. The cells are then either profiled in situ or after single cell isolation.

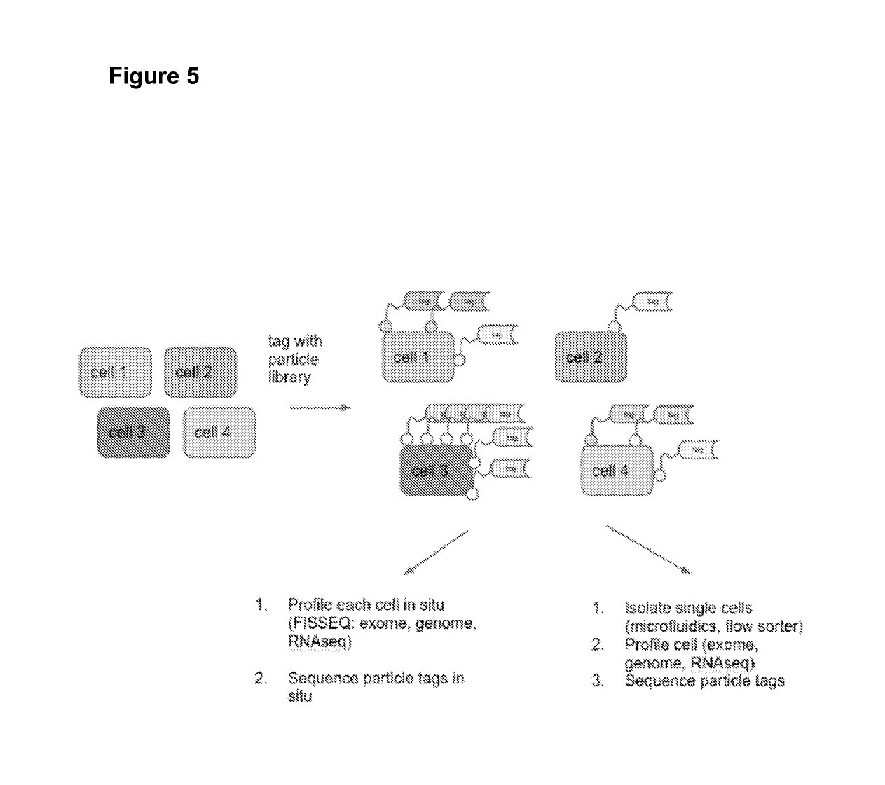

FIG. 6 shows an illustration of potential nucleic acid based approaches of attaching a nucleic acid to a particle.

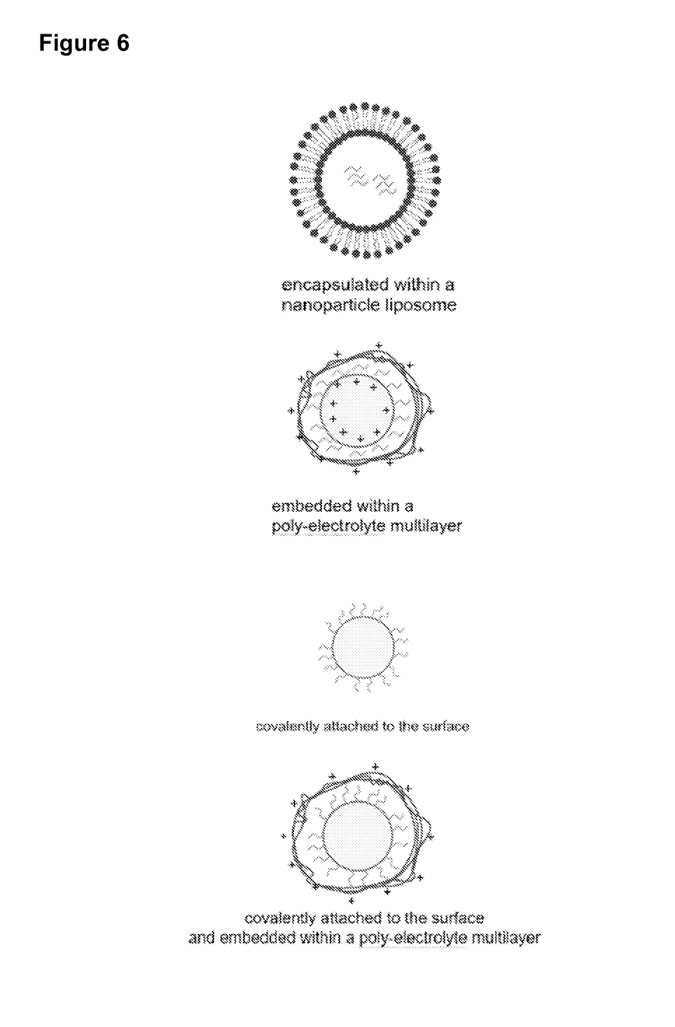

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present disclosure.

### DETAILED DESCRIPTION OF THE DISCLOSURE

The present disclosure relates to general methods of tagging a functionalized particle with a tag that uniquely identifies each type of functionalized particle. A plurality of different tagged particles can then be pooled, and this complex mixture of tagged, functionalized particles can then be introduced into an in vitro or in vivo assay to select for specific properties. Particles that demonstrate the desired property can then be isolated (e.g., by virtue of their location or concentration in the body, binding affinity, chemical properties, etc.) and their tags can be read. This facilitates identification of functionalized particles having the desired property.

In one aspect, the disclosure provides a tagged, functionalized particle comprising one or more functional moieties and a unique tag linked to a particle.

In another aspect, the disclosure provides methods of preparing a tagged, functionalized particle comprising linking one or more functional moieties and a unique tag to a particle.

As used herein, the terms “tag” and “unique tag” refer to any chemical moiety that uniquely identifies the particle to which it is linked. The tags are synthesized separately from the particles and functionalized particles (i.e., before linking the tags to the particles). The tags are not involved in the screening process (i.e., the tags serve to identify the functionalized particle, not to interact with or screen for a desired property). Tag sequences are specified (i.e., user defined) in advance of synthesizing the tagged, functionalized particles and are uniquely associated with only one particle type with known (i.e., user defined) functional moieties.

In certain embodiments, the unique tag is a nucleic acid (i.e., an oligonucleotide), wherein the oligonucleotide is 5-150 nucleotides (i.e., a barcode) of single or double stranded DNA, RNA or XNA. In some embodiments, the oligonucleotide can be about 5 to about 150 nucleotides in length, about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, or about 5 to about 10 nucleotides in length and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated. Nucleic acid sequences are designed according to standard principles to minimize secondary structure, off-target binding, and complexity (see, for example, Mir et al., PLOS ONE 8(12):e82933 (2013); Bystrykh, PLOS ONE 7(5):e36852 (2012)). Single stranded oligonucleotides can be synthesized or obtained commercially (e.g., Integrated DNA Technologies). Methods of making oligonucleotides of predetermined sequences are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991), which are incorporated by reference in their entirety.

In other embodiments the tag is a peptide, wherein the peptide is a short peptide, a peptoid, or a specific pattern of isotope labeled peptide. In such an embodiment, the peptide of peptoid can be 5-20 amino acids of known sequence.

In yet other embodiments, the tag is an optically encoded tag, wherein the optical component is one or a combination of fluorescent molecules or dyes (non-limiting examples of fluorescent molecules or dyes can be green fluorescent protein (GFP), cyan fluorescent protein (CFP), yellow fluorescent protein (YFP), blue fluorescent protein (BFP), HcRed, DsRed, mCherry, rhodamine, acridine dyes, fluorescein isothiocyanate (FITC), Alexa Fluor®488, Alexa Fluor®532, Alexa Fluor®546, Alexa Fluor®594, Alexa Fluor®633, Alexa Fluor®647, Alexa Fluor®660, Alexa Fluor®750, tetramethylrhodamine-5-(and 6)-isothiocyanate (TRITC), 6-FAM, TAMRA™, JOE, MAX, TET™, ROX, HEX, TYE™ 563, TYE™ 665, TYE™ 705, Cy2™, Cy3™, Cy5™ and Cy7™).

In other embodiments, the tag is an optically-encoded tag, wherein the optical component is one or a combination of quantum dots (quantum dots are small particles and can be a nanocrystal made of semiconductor materials that is small enough to exhibit unique quantum mechanical and/or optical properties; traditionally chalcogenides (selenides or sulfides) of metals like gold, silver, iron, cobalt, zinc, cadmium, nickel, gadolinium, chromium, copper, manganese, palladium, tin, and alloys and/or oxides thereof (CdSe, ZnO or ZnS, for example), which range from 2 to 10 nanometers in diameter), polymer dots (see Zhang et al., Angewandte Chemie Int Ed Engl. 52(15):4127-31, (2013)), nanodiamonds, and Førster resonance energy transfer/fluorescence resonance energy transfer (FRET) systems (using components with sufficient spectral separation and unique optical properties).

In yet another embodiment, the unique tag is a mass-encoded or isotopic-encoded tag, wherein the mass-encoded tag is a lanthanide atom or a peptide, either single species or combinations thereof.

The term “functional moiety” refers to any chemical or biological moiety to be tested for a desired property. In some embodiments, the functional moiety is a monoclonal antibody, a polyclonal antibody, single chain antibody, a single domain antibody or nanobody, a bi-specific antibody, an affibody molecule, a peptide, a peptoid, a small molecule (which is typically, but not always, an organic compound of less than about 1000 Da, but may include such an organic compound complexed or chelated with a metal), an aptamer or other nucleic acid, or other chemical compound. A functional moiety can exist in a library of functional moieties. Non-limiting examples of libraries include libraries of synthetic small molecules, natural products or extracts, and purified enzymes such as proteases or kinases. In some embodiments, the functional moiety is one that cannot be synthesized on a single solid support. Representative examples of such functional moieties are antibodies and polymers (other than, for example, nucleic acid and amino acid polymers).

In one embodiment, the functional moiety is an antibody. As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” refers to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids and more typically comprises at least about 200 amino acids. Antibodies to many markers are known to those of skill in the art and can be obtained commercially or readily produced by known methods such as using phage-display or yeast-display technology.

In another embodiment, the functional moiety is an aptamer. Sometimes referred to as “synthetic antibodies,” aptamers are pre-selected single-stranded oligonucleotide (e.g., DNA or RNA) or peptide molecules that bind to specific target molecules including proteins and peptides with affinities and specificities that are comparable to antibodies. These molecules can assume a variety of shapes due to their propensity to form helices and single-stranded loops with specific binding pockets, explaining their versatility in binding to diverse targets. Their specificity and characteristics are not directly determined by their primary sequence but by their tertiary structure which is analogous to the globular shape of tRNA. Aptamers have a wide range of applications including diagnostics and therapeutics and can be chemically synthesized using known techniques. Furthermore, aptamers can offer a number of advantages over traditional antibodies including avoiding the need to specifically know the precise epitopes or biomarkers themselves. Finally, aptamers are typically non-immunogenic, easy to synthesize, characterize, modify and exhibit high specificity and affinity for their target antigen.

By using a variety of selection techniques, aptamers can be selected to find targets, e.g., on a surface or inside a cell of interest or in a bodily fluid, without the need to identify the precise biomarker or epitopes themselves. In many cases, the aptamer identification process can begin with a large random pool of oligonucleotides or peptides that are systematically subjected to iterative negative and positive rounds of selection against a target, e.g., a protein molecule, to separate out low affinity or unspecific binders. The remaining aptamers in the enriched pool can be collected and propagated, e.g., PCR amplified, and used in subsequent rounds of selection. Typically anywhere from three to twenty cycles of target binding, separation, and amplification are carried out and the candidate aptamers are then characterized for binding affinity and specificity. This selection process, referred to as Systemic Evolution of Ligands by Exponential Enrichment or SELEX, is commonly used for selecting and identifying highly-targeted aptamers directed to a wide variety of targets include whole living cells. For a review of SELEX methods to screen and separate binding molecules, e.g., aptamers, from libraries of aptamers, see Stoltenburg et al. Biomolecular Engineering, 2007, Vol. 24, pp. 381-403; and Ozer et al., Molecular Therapy Nucleic Acids, 2014, Vol. 3, e183, published on line Aug. 5, 2014, both which are incorporated by reference in their entirety. Various methods have been used for separating out the target bound and unbound aptamers including nitrocellulose filter binding, bead-based, electrophoretic, microfluidic, microarray-based, and microscopic.

In some embodiments, the functional moieties bind to a target analyte, e.g. glucose or ion such as sodium, potassium, calcium, or chloride) in a bodily fluid such as blood, interstitium, or perspiration. In other embodiments, functional moieties bind to an organ, tissue, cell, extracellular matrix component, and/or intracellular compartment that is associated with a specific developmental stage or a specific disease state (i.e. a “target” or “marker”). In some embodiments, a target is an antigen on the surface of a cell, such as a cell surface receptor, an integrin, a transmembrane protein, an ion channel, and/or a membrane transport protein. In some embodiments, a target is an intracellular protein. In some embodiments, a target is a soluble protein, such as immunoglobulin. In some embodiments, a target is more prevalent, accessible, and/or abundant in a diseased locale (e.g. organ, tissue, cell, subcellular locale, and/or extracellular matrix component) than in a healthy locale.

The term “particle” refers to any chemical or physical moiety that serves as a substrate or carrier for one or more functional moieties to which the unique tag can also be linked. The particle facilitates associating a tag with a functional moiety or moieties and can assist in the isolation or identification of the tag linked to a functionalized particle. In some embodiments, the particles have detectable optical and/or magnetic properties. In an embodiment, an optically detectable particle is one that can be detected within a living cell using optical means compatible with cell viability. In some embodiments, particles can have unique optical (i.e., fluorescent), mechanical, and thermal properties; and are non-toxic. Particles can include, for example, and without limitation, a metal, a semiconductor, and an insulator particle composition, and a polymer (linear, branched, dendrimer (organic and inorganic)). Additional examples of particles include, without limitation, quantum dots, plasmonic particles such as gold or silver particles, upconverting nanocrystals, iron oxide particles or other superparamagnetic or magnetic particles, silica, liposomes, micelles, carbon nanotubes, doped or undoped graphene, graphene oxide, nanodiamonds, titania, alumina, and metal oxides. In some embodiments, particles can be optically or magnetically detectable. In some embodiments, intrinsically fluorescent or luminescent particles, particles that comprise fluorescent or luminescent moieties, plasmon resonant nanoparticles, and magnetic particles are among the detectable particles that are used in various embodiments. In general, the particles should have dimensions small enough to be functional, but not toxic or detrimental to the subject when injected into a subject. Typically the particles can have a longest straight dimension (e.g., diameter) of less than 1000 nm, 900 nm, 800 nm, 700 nm, 600 nm, 500 nm, 400 nm, 300 nm, 200 nm or less. In some embodiments, the particles can have a diameter of 200 nm or less. In other embodiments, the particles have a diameter of 100 nm or less. Smaller particles, e.g. having diameters of 50 nm or less, e.g., 5 nm-30 nm, are used in some embodiments. In an embodiment, the particle size is 50 nm-300 nm.

The optical property can be a feature of absorption, emission, or scattering spectrum or a change in a feature of an absorption, emission, or scattering spectrum. The optical property can be a visually detectable feature such as, for example, color, apparent size, or visibility (i.e. simply whether or not the particle is visible under particular conditions). Features of a spectrum include, for example, peak wavelength or frequency (wavelength or frequency at which maximum emission, scattering intensity, extinction, absorption, etc. occurs), peak magnitude (e.g., peak emission value, peak scattering intensity, peak absorbance value, etc.), peak width at half height, or metrics derived from any of the foregoing such as ratio of peak magnitude to peak width. Certain spectra may contain multiple peaks, of which one is typically the major peak and has significantly greater intensity than the others. Each spectral peak has associated features. Typically, for any particular spectrum, spectral features such as peak wavelength or frequency, peak magnitude, peak width at half height, etc., are determined with reference to the major peak. The features of each peak, number of peaks, separation between peaks, etc., can be considered to be features of the spectrum as a whole. The foregoing features can be measured as a function of the direction of polarization of light illuminating the particles; thus polarization dependence can be measured. Features associated with hyper-Rayleigh scattering can be measured. Fluorescence detection can include detection of fluorescence modes. Luminescence detection can also be useful for optical imaging purposes. Raman scattering can also be useful as well.

In some embodiments, the particles can be biocompatible and/or biodegradable. As used herein, the term “biocompatible” refers to substances that are not toxic to cells or are present in levels that are not toxic to cells. In some embodiments, a substance is considered to be “biocompatible” if its addition to cells in vivo does not induce inflammation and/or other adverse effects in vivo. In other embodiments, the materials composing the functionalized particles can be generally recognized as safe (GRAS) or FDA-approved materials. In general, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

In some embodiments, the particle comprises a polymer matrix or a bead. Beads are known in the art and available from various manufacturers. Non-limiting examples of beads that can be used are magnetic beads (magnetic beads to magnetically responsive particles that contain one or more metals or oxides or hydroxides thereof, examples include, but are not limited to COMPEL™, PROMAG™, Dynabeads®, ADEMTECH, Chemicell), agarose beads (Sepharose®), polystyrene beads, polyethylene microsphere beads, and beads composed at least in part of polymethylmethacrylate, polyacrylamide, polyethylene glycol, poly(vinyl chloride), carboxylated poly(vinyl chloride), or poly(vinyl chloride-co-vinyl acetate-co-vinyl alcohol) may be used. In some embodiments, the particle comprises diamond nanoparticles. Diamond nanoparticles are typically about 5 nm in size offer a large accessible surface and tailorable surface chemistry.

In some embodiments, the particles have detectable optical and/or magnetic properties. In various embodiments, intrinsically fluorescent or luminescent particles, particles that comprise fluorescent or luminescent moieties, plasmon resonant particles, and magnetic particles are among the detectable particles that can be used. Such particles can have a variety of different shapes including variety of different shapes including spheres, oblate spheroids, cylinders, ovals, ellipses, shells, cubes, cuboids, cones, pyramids, rods (e.g., cylinders or elongated structures having a square or rectangular cross-section), tetrapods (nanoparticles having four leg-like appendages), triangles, prisms, etc. Particles can be also solid or hollow and can comprise one or more layers (e.g., nanoshells, nanorings, etc.). Particles may have a core/shell structure, wherein the core(s) and shell(s) can be made of different materials. Particles may comprise gradient or homogeneous alloys. Particles may be a composite made of two or more materials, of which one, more than one, or all of the materials possess magnetic properties, electrically detectable properties, and/or optically detectable properties.

The term “functionalized particle” refers to a particle to which one or more functional moieties are associated (e.g., covalently or otherwise linked). The functionalized particles are synthesized in a manner such that the identity of the tag and the functional moiety, and their correspondence to each other, are determined and known prior to assembly of the tagged, functionalized particle (i.e., the functionalized particles are linked with unique tags to comprise tagged, functionalized particles). In some embodiments, the tagged, functionalized particles and libraries of tagged, functionalized particles comprise tagged, functionalized particles that are not complexed to or otherwise comprise a target or binding partner and libraries of them are not complexed to or otherwise comprise a target or binding partner.

The term “linking” or “linked” refers to any method of uniquely associating, conjugating or attaching a tag or functional moiety with a particle. For example, a tag could be embedded non-covalently within or covalently to a particle polymer matrix (see, for example, Poon et al., Nano Lett. 11:2096-2103, (2011)). In another non-limiting example, a tag could be covalently attached to the surface of the particle (see, for example, Margulies et al., Nature 437:376-380, (2005)). The only requirement with respect to “linking” is that the tag remains associated with functionalized particle under the experimental conditions (e.g., assay conditions) to which the tagged, functionalized particle is subject and accessible to detection and identification. For example, linkers contemplated include linear polymers (e.g., polyethylene glycol, polylysine, dextran, etc.), branched-chain polymers (see, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993, all which are incorporated by reference in their entirety); lipids; cholesterol groups (such as a steroid); or carbohydrates or oligosaccharides. Other linkers include one or more water soluble polymer attachments such as polyoxyethylene glycol, or polypropylene glycol as described U.S. Pat. Nos. 4,640,835; 4,496,689; 4,301,144; 4,670,417; 4,791,1921 and 4,179,337, all which are incorporated by reference in their entirety. Other useful polymers as linkers known in the art include monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers. In some embodiments, cleavable linkers can be used to conjugate the functional moiety or the unique tag to the particle. In other embodiments, the linkers are non-cleavable.

Linkers can be functional groups or reactive groups or may include functional or reactive groups. Functional groups include monofunctional linkers comprising a reactive group as well as multifunctional crosslinkers comprising two or more reactive groups capable of forming a bond with two or more different functional targets (e.g., labels, proteins, macromolecules, semiconductor nanocrystals, or substrate). In some embodiments, the multifunctional crosslinkers are heterobifunctional crosslinkers comprising two or more different reactive groups. Suitable reactive groups include, but are not limited to thiol (—SH), carboxylate (—COO), carboxylic acid (—COOH), amine (NH₂), hydroxyl (—OH), aldehyde (—CHO), alcohol (ROH), ketone (R₂CO), active hydrogen, ester, sulfhydryl (SH), phosphate (—PO₃), photoreactive moieties, azides, alkynes, alkenes, or tetrazines. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

Other suitable reactive groups and classes of reactions include those that are well known in the art of bioconjugate chemistry. Currently favored classes of reactions available with reactive chelates are those which proceed under relatively mild conditions. These include, but are not limited to, nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March (1985) Advanced Organic Chemistry, 3rd Ed., John Wiley & Sons, New York, Hermanson (1996) Bioconjugate Techniques, Academic Press, San Diego; and Feeney et al. (1982) Modification of Proteins; Advances in Chemistry Series, Vol. 198, American Chemical Society, Washington, D.C., which are incorporated by reference in their entirety.

In certain embodiments, a collection of tagged, functionalized particles is prepared by pooling two or more types of tagged, functionalized particle, wherein each type of functionalized particle comprises a unique combination of tag and functional moiety or moieties. For example, a collection of tagged, functionalized particles could comprise a plurality (e.g., 100) types of functionalized particles, each type comprising a particular antibody or antibodies and tag. In another embodiment, the types of tagged, functionalized particles in the collection of functionalized particles include particles in which one of a plurality of antibodies (e.g., one of 100) and one of a plurality of small molecules (e.g., one of 100 small molecules). In such an embodiment in which there were 100 different antibodies and 100 small molecules, the collection would comprise 10,000 different functionalized particles, each uniquely tagged to identify the particular combination of antibody and small molecule associated with the tagged particle, spanning all possible combinations.

In another aspect, the disclosure provides a method of screening a collection of uniquely tagged, functionalized particles to identify a functional moiety or combination of functional moieties having a desired property or properties, the method comprising introducing the collection of tagged, functionalized particles into an assay to select for a specific property or properties; isolating the tagged, functionalized particles that manifest the desired property or properties; identifying the tags of the isolated tagged, functionalized particles; and determining the functional moiety or combination of functional moieties from the identity of the tag.

In some embodiments, the assay is in vitro or in vivo, and the desired property is a location, a concentration, a binding affinity, a pharmacokinetic, a pharmacodynamic, or a chemical property of the functional moiety or combination of functional moieties. Non-limiting examples of assays include isolated molecular target assays, cell-free multicomponent assays, and cell- or organism-based assays. In some embodiments, the assay screens for the ability of a pool or collection of functionalized particles to bind to an engineered or patient-derived cell line or tissue. Generally, screening is conducted while the functional moiety (e.g., a ligand) remain attached to the particles.

In yet another embodiment, the unique tags are read by methods one of skill in the art would recognize as appropriate according to the unique tag used. For example, nucleotide sequencing could be used if the tags are nucleic acids; mass spectrometry if the tags are peptides or mass-encoded or isotopic-coded; or flow cytometry or microscopy if the tags are fluorescent dyes, quantum dots or nanodiamonds.

The nucleotide sequencing technique used in the methods described herein can generate, for example, about 30 bp, about 40 bp, about 50 bp, about 60 bp, about 70 bp, about 80 bp, about 90 bp, about 100 bp, about 110, about 120 bp per read, about 150 bp, about 200 bp, about 250 bp, about 300 bp, about 350 bp, about 400 bp, about 450 bp, about 500 bp, about 550 bp, or about 600 bp per read. Nucleotide sequencing techniques can include, but are not limited to Maxam-Gilbert sequencing, chain-termination methods, next generation methods (for example, massively parallel signature sequencing (MPSS), polony sequencing, 454 pyrosequencing, Illumina (Solexa) sequencing, SOLiD sequencing, ion torrent semiconductor sequencing, DNA nanoball sequencing, heliscope single molecule sequencing, single molecule real time (SMRT) sequencing), nanopore DNA sequencing, sequencing by hybridization, sequencing with mass spectrometry, microfluidic Sanger sequencing or in vitro virus high-throughput sequencing.

As each functionalized particle comprises a unique tag for the functional moiety or combination of functional moieties associated with the particle, after the tag is read (i.e., identified), the specific functional moiety or combination of functional moieties can be identified.

The methods described herein can be used to identify a wide variety of molecules that can be used for diagnostic systems, pharmaceutical purposes, cosmetic, and medicinal purposes that are known in the art. Furthermore, the methods can allow a researcher to quickly conduct millions of chemical, genetic, or pharmacological tests. Through this process one can rapidly identify active compounds, antibodies, or genes that modulate a particular biomolecular pathway.

A diagnostic system can non-invasively detect and measure a plurality of physiological parameters of a subject, which can include any parameters that may relate to the subject's health. For example, the system could include sensors for measuring blood pressure, pulse rate, skin temperature, etc. At least some of the physiological parameters may be obtained by the system non-invasively detecting and/or measuring one or more analytes in blood circulating in subsurface vasculature. The one or more analytes could be any analytes that, when present in or absent from the blood, or present at a particular concentration or range of concentrations, may be indicative of a medical condition or health of the person. For example, the one or more analytes could include ions such as sodium potassium, calcium, and chloride, enzymes, hormones, proteins, drug metabolites, tumor cells, tumor markers or other molecules.

In an example embodiment, the system obtains at least some of the health-related information by detecting the binding or interaction of a clinically-relevant analyte to or with materials such as functionalized particles, introduced into a lumen of the subsurface vasculature that have been functionalized with a targeting entity that has a specific affinity to bind to or interact with the specific analyte such as glucose. The term “binding” is understood in its broadest sense to also include a detectable interaction between the clinically relevant analyte and the tagged, functionalized particles. The tagged, functionalized particles can be introduced into the subject's blood stream by injection, ingestion, inhalation, transdermally, or in some other manner.

The tagged, functionalized particles can be functionalized by covalently or otherwise attaching or associating a targeting entity that specifically binds, undergoes cell uptake or otherwise interacts with a particular clinically-relevant target analyte with a defined affinity to the target analyte. Other compounds or molecules, such reporter labels, e.g., fluorophores or autofluorescent or luminescent markers or non-optical contrast agents (e.g. acoustic impedance contrast, RF contrast and the like), which may assist in interrogating the functionalized particles in vivo, may also be attached to the particles.

The tagged, functionalized particles include can comprise nanoparticles having a diameter that is generally equal to or less than about 200 micrometers. In some embodiments, the nanoparticles have a diameter on the order of about 10 nanometers to 1 micrometer. In further embodiments, small nanoparticles on the order of 10-100 nanometers in diameter may be assembled to form a larger “clusters” or “assemblies on the order of 1-10 micrometers. Further, a nanoparticle may be of any shape, for example, spheres, rods, non-symmetrical shapes, etc.

The system may further include one or more data collection systems for interrogating, in a non-invasive manner, the tagged, functionalized particles present in a lumen of the subsurface vasculature in a particular local area. In one example, the system includes a detector configured to detect a response signal transmitted from a portion of subsurface vasculature. The response signal can include both an analyte response signal, which can be related to the interaction of the one or more target analytes with the tagged, functionalized particles, and a background noise signal. For example, the tagged, functionalized particles may include a chemiluminescent marker configured to produce a response signal in the form of luminescence radiation produced in response to a chemical reaction initiated, at least in part, to the binding of the target analyte to the particle.

In some examples, the system may also include an interrogating signal source for transmitting an interrogating signal that can penetrate into a portion of subsurface vasculature, or another body system, and a detector for detecting a response signal that is transmitted from the portion of subsurface vasculature, or other body system, in response to the interrogating signal. The interrogating signal can be any kind of signal that is benign to the patient, such as electromagnetic, magnetic, optic, acoustic, thermal, mechanical, electric and results in a response signal that can be used to measure a physiological parameter or, more particularly, that can detect the binding or interaction of the clinically-relevant analyte to the tagged, functionalized particles. In one example, the interrogating signal is a radio frequency (RF) signal and the response signal is a magnetic resonance signal, such as nuclear magnetic resonance (NMR). In another example, where the tagged, functionalized particles include a fluorophore, the interrogating signal is an optical signal with a wavelength that can excite the fluorophore and penetrate the skin or other tissue and subsurface vasculature (e.g., a wavelength in the range of about 500 to about 1000 nanometers), and the response signal is fluorescence radiation from the fluorophore that can penetrate the subsurface vasculature and tissue to reach the detector. In another example, where the tagged, functionalized particles include an electrically conductive material or a magnetically lossy material, the interrogation signal may be a time-varying magnetic field or a radio frequency (RF) electromagnetic signal, with sufficient signal power to rapidly heat the particles. The response signal may be an acoustic emission from the particles, caused by rapid thermal expansion of the particles, or caused by cavitation of the liquid medium in contact with the particles. As described above, in some cases, an interrogating signal may not be necessary to produce an analyte response signal.

Additionally, the system may further include a modulation source configured to modulate the analyte response signal. The modulation source can be configured to modulate the analyte response signal differently than the background noise signal. To this end, the modulation may help to discern between the target analyte and, essentially, everything else in the body by, for example, increasing the signal-to-noise ratio. Generally, the modulation may include any spatial, temporal, spectral, thermal, magnetic, mechanical, electrical, acoustic, chemical, or electrochemical, etc. modulation technique or any combination thereof.

In some scenarios, it may also be useful to detect and distinguish both the analyte response signal—related to tagged, functionalized particles bound to or interacting with target analyte(s)—and an “unbound” particle signal—related to functionalized particles not bound to or interacting with target analyte(s). For example, in some measurement or characterization schemes, it may be useful to determine the percentage of tagged, functionalized particles introduced into the body that have bound to the target analyte. In such cases, the modulation source may be configured to modulate the analyte response signal differently than the unbound particle signal.

Data collected by the detector may be sent to a processor for analysis. The processor may be configured to non-invasively detect the one or more target analytes by differentiating the analyte response signal from the background noise signal based, at least in part, on the modulation. In some cases, the processor may further be configured to differentiate the analyte response signal from the unbound particle signal. Further, the processor may be configured to determine the concentration of a particular target analyte in the blood from, at least in part, the analyte response signal. The detection and concentration data processed by the processor may be communicated to the patient, transmitted to medical or clinical personnel, locally stored or transmitted to a remote server, the cloud, and/or any other system where the data may be stored or accessed at a later time.

The processor may be located on an external reader, which may be provided as an external body-mounted device, such as a necklace, wristwatch, eyeglasses, a mobile phone, a handheld or personal computing device or some combination thereof. Data collected by the detector may be transmitted to the external reader via a communication interface. Control electronics can wirelessly communicate the data to the external reader by modifying the impedance of an antenna in communication with the detector so as to characteristically modify the backscatter from the antenna. In some examples, the external reader can operate to intermittently interrogate the detector to provide a reading by radiating sufficient radiation to power the detector to obtain a measurement and communicate the result. In this way, the external reader can acquire a series of analyte identification and concentration measurements over time without continuously powering the detector and/or processor. The processor may also be provided at another location distal to the detector, and the detector data is communicated to the processor via a wired connection, a memory card, a USB device or other known method. Alternatively, the processor may be located proximal to the detector and may be configured to locally analyze the data that it collects and then transmit the results of the analysis to an external reader or server.

The external reader may include a user interface, or may further transmit the collected data to a device with a user interface that can indicate the results of the data analysis. In this way, the person wearing, holding or viewing the device can be made aware of the analysis and/or potential medical conditions. The external reader may also be configured to produce an auditory or tactile (vibration) response to alert the patient of a medical condition. Further, the external reader may also be configured to receive information from the patient regarding his/her health state, wellness state, activity state, nutrition intake and the like, as additional input information to the processor. For example, the user may input a health or wellness state, such as, experiencing migraine symptoms, jittery, racing heart, upset stomach, feeling tired, activity state including types and duration of physical activity nutrition intake including meal timing and composition, and other parameters including body weight, medication intake, quality of sleep, stress level, personal care products used, environmental conditions, social activity, etc. Further, the reader may also receive signals from one or more other detectors, such as a pedometer, heart rate sensor, blood pressure sensor, blood oxygen saturation level, body temperature, GPS or other location or positioning sensors, microphone, light sensor, etc.

The system may be configured to obtain data during pre-set measurement periods or in response to a prompt. For example, the system may be configured to operate the detector and collect data once an hour. In other examples, the system may be configured to operate the detector in response to a prompt, such as a manual input by the patient or a physician. The system may also be configured to obtain data in response to an internal or external event or combination of events, such as during or after physical activity, at rest, at high pulse rates, high or low blood pressures, cold or hot weather conditions, etc. In other examples, the system could operate the detector more frequently or less frequently, or the system could measure some analytes more frequently than others.

Data collected by the system may be used to notify the patient of, as described above, analyte levels or of an existing or imminent medical emergency. In some examples, the data may be used to develop an individual baseline profile for the patient. The baseline profile may include patterns for how one or more of the patient's analyte levels typically change over time, such as during the course of a day, a week, or a month, or in response to consumption of a particular type of food/drug. The baseline profile, in essence, may establish “normal” levels of the measured analytes for the patient. Additional data, collected over additional measurement periods, may be compared to the baseline profile. If the additional data is consistent with the patterns embodied in the baseline profile, it may be determined that the patient's condition has not changed. On the other hand, if the additional data deviates from the patterns embodied in the baseline profile, it may be determined that the patient's condition has changed. The change in condition could, for example, indicate that the patient has developed a disease, disorder, or other adverse medical condition or may be at risk for a severe medical condition in the near future. Further, the change in condition could further indicate a change in the patient's eating habits, either positively or negatively, which could be of interest to medical personnel. Further, the patient's baseline and deviations from the baseline can be compared to baseline and deviation data collected from a population of wearers of the devices.

When a change in condition is detected, a clinical protocol may be consulted to generate one or more recommendations that are appropriate for the patient's change in condition. For example, it may be recommended that the patient inject himself/herself with insulin, change his/her diet, take a particular medication or supplement, schedule an appointment with a medical professional, get a specific medical test, go to the hospital to seek immediate medical attention, abstain from certain activities, etc. The clinical protocol may be developed based, at least in part, on correlations between analyte concentration and health state derived by the server, any known health information or medical history of the patient, and/or on recognized standards of care in the medical field. The one or more recommendations may then be transmitted to the external reader for communication to the user via the user interface.

Correlations may be derived between the analyte concentration(s) measured by the system and the health state reported by the patient. For example, analysis of the analyte data and the health state data may reveal that the patient has not responded to chemotherapy when an analyte reaches a certain concentration. This correlation data may be used to generate recommendations for the patient, or to develop a clinical protocol. Blood analysis may be complemented with other physiological measurements such as blood pressure, heart rate, body temperature etc., in order to add to or enhance these correlations.

Further, data collected from a plurality of patients, including both the analyte measurements and the indications of health state, may be used to develop one or more clinical protocols used by the server to generate recommendations and/or used by medical professionals to provide medical care and advice to their patients. This data may further be used to recognize correlations between blood analytes and health conditions among the population. Health professionals may further use this data to diagnose and prevent illness and disease, prevent serious clinical events in the population, and to update clinical protocols, courses of treatment, and the standard of care.

The above described system may be implemented as a device. In one embodiment, the device is a wearable device. The term “wearable device,” as used in this disclosure, refers to any device that is capable of being worn at, on or in proximity to a body surface, such as a wrist, ankle, waist, chest, ear, eye or other body part. In order to take in vivo measurements in a non-invasive manner from outside of the body, the wearable device may be positioned on a portion of the body where subsurface vasculature is easily observable, the qualification of which will depend on the type of detection system used. The device may be placed in close proximity to the skin or tissue, but need not be touching or in intimate contact therewith. A mount, such as a belt, wristband, ankle band, headband, etc. can be provided to mount the device at, on or in proximity to the body surface. The mount may prevent the wearable device from moving relative to the body to reduce measurement error and noise. Further, the mount may be an adhesive substrate for adhering the wearable device to the body of a wearer. The detector, modulation source, interrogation signal source (if applicable) and, in some examples, the processor, may be provided on the wearable device. In other embodiments, the above described system may be implemented as a stationary measurement device to which a user must be brought into contact or proximity with or as a device that may be temporarily placed or held against a body surface during one or more measurement periods.

It should be understood that the above embodiments, and other embodiments described herein, are provided for explanatory purposes, and are not intended to be limiting.

All publications, patents and patent applications cited herein are hereby expressly incorporated by reference for all purposes to the extent they are consistent with this disclosure.

Methods well known to those skilled in the art can be used to construct expression vectors and recombinant bacterial cells according to this disclosure. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “nucleic acid”, “polynucleotide”, “nucleotide”, and “oligonucleotide” can be used interchangeably to refer to single stranded or double stranded, nucleic acid comprising DNA, RNA, derivatives thereof (e.g., xenonucleic acids (XNA), made with synthetic nucleic acids and capable of information storage like DNA or RNA), or combinations thereof.

### EXAMPLES

The Examples that follow are illustrative of specific embodiments of the invention, and various uses thereof They are set forth for explanatory purposes only, and are not to be taken as limiting the invention.

### Example 1

### Nucleic Acid Based Approaches

DNA (single or double stranded) or RNA, or XNA (xenonucleic acids, made with synthetic nucleic acids and capable of information storage like DNA or RNA) molecules containing a unique sequence (i.e., a unique tag) could be embedded non-covalently or covalently within the particle polymer matrix or core. For example, the negatively charged nucleic acids can be incorporated within a poly-electrolyte multilayer (i.e., a layer-by-layer system; see Poon et al., Nano Lett. 11:2096-2103, (2011)). Alternatively, nucleic acids could be covalently attached to the surface of the particle or hybridized to a complementary sequence attached to the surface of the particle. The surface can be functionalized with appropriate chemical groups to bind (covalently or non-covalently) with groups on the nucleic acid. For example, —COOH groups on the surface reacting with —NH₂ functionalized DNA; or streptavidin on the surface binding to biotinylated DNA; or a common nucleic acid sequence on the surface facilitating hybridization to a reverse complement on the barcode (see Margulies et al., Nature 437:376-380, (2005)).

After particle selection in vitro or in vivo, the nucleic acids could be recovered and sequenced on a massively parallel sequencer to reveal the tag identities and relative abundance, along with the corresponding particles' identities and properties. Nucleic acid recovery can require appropriate chemical and/or enzymatic conditions to remove any outer protecting layers (e.g. trypsin to degrade a poly-L-lysine protecting layer), or removal of the covalent or non-covalent links, and elution in appropriate solution.

Quantitative PCR could also be used as way to read out the relative quantities of a subset of the tags through the use of barcode specific PCR.

### Nucleic Acid Design

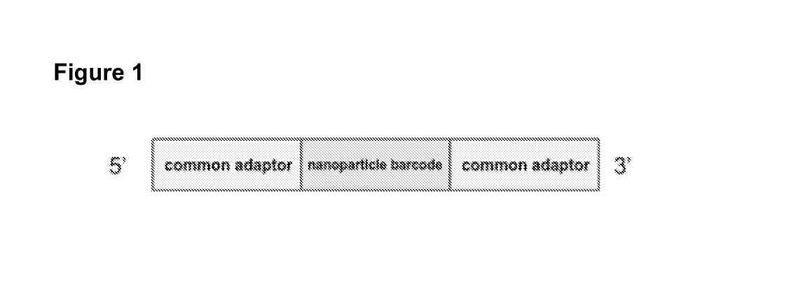

Barcode sequences are designed according to standard principles to minimize secondary structure, off-target binding, and complexity (see, for example, Mir et al., PLOS ONE 8(12):e82933 (2013); Bystrykh, PLOS ONE 7(5):e36852 (2012)). A single stranded oligonucleotide could be synthesized commercially (e.g. using Integrated DNA Technologies) as shown in FIG. 1.

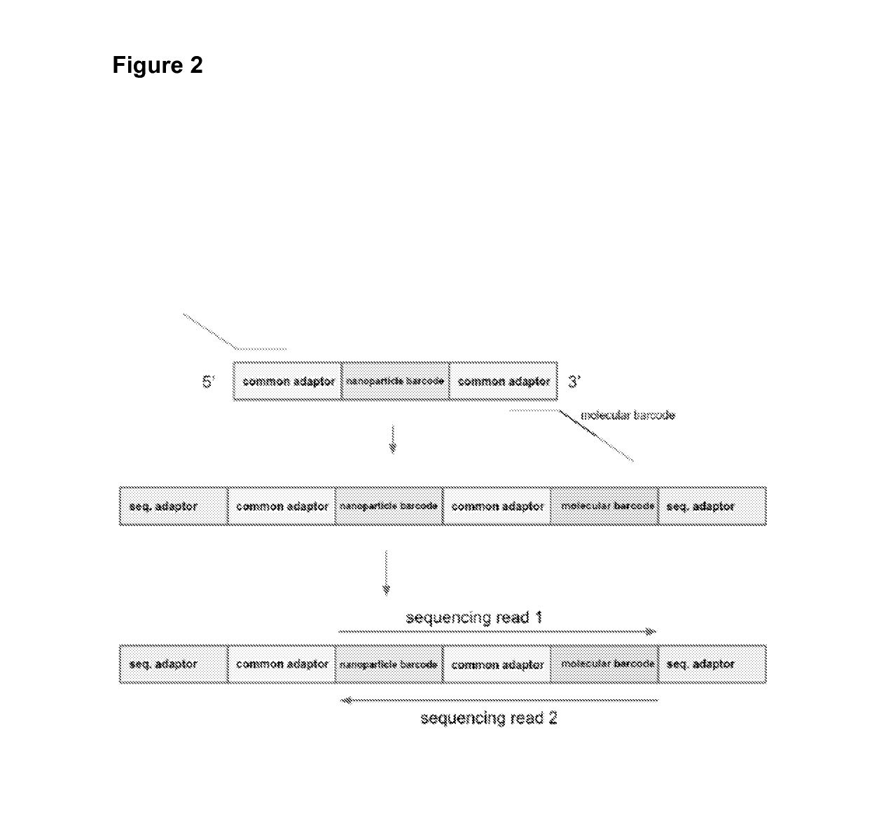

The tagged, functionalized particles barcode can be comprised of 6-25 nucleotides of known sequence and is flanked by common adaptor sequences (the number of unique barcodes is 4̂n, where n is the length of the barcode). This molecule can be annealed to its reverse complement to make a double stranded construct, or used as single stranded. Following incorporation of the oligonucleotide into or on the particle as described above, specific barcoded particles can be further functionalized with additional coatings, targeting groups, stealth layers, etc. through standard chemistries (see, for example, Wang & Thanou, Pharma. Res. 6(2):90-99 (2010); Weissleder et al., Nature Biotech, 23(11):1418-1423 (2005), and Davis et al., Nature 464:1067-1071 (2010)). Different particle types can then be pooled and subjected to in vitro or in vivo cytometry to select for particles with certain properties. After selection, the nucleic acid can be amplified as shown in FIG. 2. This amplification can occur either on the particle surface or following release from the particle surface (or directly on the particle surface).

In current experiments, the forward primer: (AATGATACGGCGACCACCGAGATCTACACTCTTTCCCTACACGACGCTCTTCCGATC T; SEQ ID NO: 01) contains the Illumina p5 sequencing adaptor (AATGATACGGCGACCACCGAGA; SEQ ID NO: 02), and the reverse primer (CAAGCAGAAGACGGCATACGAGATNNNNNNNNNNGTGACTGGAGTTCAGACGTGT GCTCTTCCGATCT; SEQ ID NO:03) contains the Illumina p7 adaptor (CAAGCAGAAGACGGCATACGAGAT; SEQ ID NO: 04). Also within the reverse primer is a second “molecular” barcode. It is also possible to include a molecular barcode in the forward primer, e.g. in SEQ ID NO:01. A dual molecular barcode modality would eliminate chimeras and improve counting statistics. It is also possible to use fixed molecular tag sequences instead of degenerate (NNNNNNNN; SEQ ID NO:06) sequences, where the tags are designed and known in advance. The molecular barcode can serve one of two purposes: first, by making it a completely or partially degenerate sequence, it can serve as a molecular tag of the barcode molecule. This will improve subsequent counting statistics by enabling the collapse of reads with common molecular barcodes into a single read, thereby eliminating PCR amplification bias. Second, molecular tags can be chosen to enable multiplexing experiments with the same set of barcodes. For example, one could perform one experiment with a set of particle barcodes and add molecular tag “A” to all barcodes, and then do a second experiment with the same barcodes and add molecular tag “B” to all barcodes. The two amplification products can then be pooled and sequenced together, and the molecular tag will identify the source of the read.

Note that the barcode construct does not necessarily have to be made using traditional chemical synthesis; it could also be made sequentially using ligation, chain extension, or hybridization based approaches.

### Results

One-hundred ninety-two different barcodes were synthesized with the following general sequence:

(SEQ ID NO: 05)
5′ ACACGACGCTCTTCCGATCT NNNNNNNN AGATCGGAAGAGCACA
CGTCTGAA 3′

In this example, NNNNNNNN (SEQ ID NO: 06) is the unique tag comprising an 8 nucleotide barcode sequence. Barcodes were loaded onto ADEMTECH 200 nm carboxylated nanoparticles using a polyelectrolyte multi-layering protocol:

### Initial Layering

1. 300 uL of ADEMTECH 200 nm carboxylated nanoparticles were placed in a 1.5 mL eppendorf tube.
2. Washed 3× with 1 mL RNAse-free DNAse-free water.
3. Resuspended in 300 uL RNAse-free DNAse-free water.
4. Added 250 uL ˜7.5 mg/mL polyLarginine (in water).
5. Placed on horizontal shaker and shake at 1500 rpm, RT, for 4 hours.
6. Washed 3× with 1 mL RNAse-free DNAse-free water.
7. Resuspended in 300 uL RNAse-free DNAse-free water.
8. Measured size/zeta potential on the ZETASIZER.
9. Repeated step 4 to 8 to add an additional layer of polyLarginine as needed.

### Barcode Loading

10. To 50 uL of this particle mixture, added 5 uL of 100 uM single-stranded DNA barcode and 50 uL of 2.5M NaCl/20% PEG buffer.
11. Placed on horizontal shaker and shake at 1500 rpm, RT, for 14 hours.
12. Washed particles 3× with 1 mL of 2.5M NaCl/20% PEG buffer. Save supernatants for subsequent qPCR analysis if needed.
13. Resuspended particles in 300 uL 2.5M NaCl/20% PEG buffer.
14. Measured size/zeta potential on the ZETASIZER (1 uL diluted to 1 mL in MilliQ H2O).

Particles can then be capped with an additional polymer layer to minimize the release of the barcode from the surface and/or to enable surface functionalization with a targeting or stealth agent. An example procedure for additional layering/layer removal can consist of:

### Capping (PLA)

15. Add 250 uL-7.5 mg/mL polyLarginine (in nuclease-free water).
16. Place on horizontal shaker and shake at 1500 rpm, RT, for 4 hours.
17. Wash 3× with 1 mL of nuclease-free water.

### Trypsin Degradation of Outer PLA Layer

18. Add 50 uL of 5 ng/uL trypsin.
19. Place on horizontal shaker and shake at 1500 rpm, 37 C for 12 hours.
20. Wash 3× with 1 mL of 2.5M NaCl/20% PEG buffer.

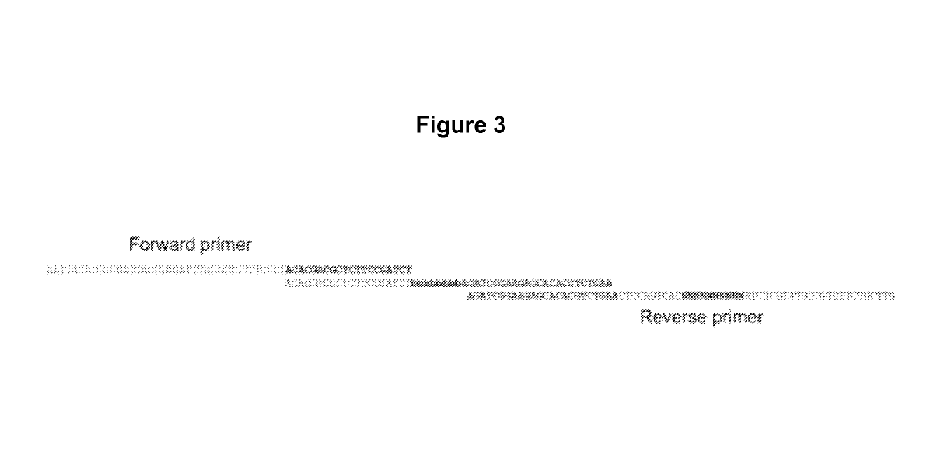

192 single stranded DNA barcodes (split into two groups of 96) were loaded onto 192 different aliquots of nanoparticles following the above protocol. Following loading, equal quantities of nanoparticles from each aliquot were pooled into two pools (each pool has 96 different types of nanoparticles). The solutions were diluted by a factor of 10̂6 and the barcodes were amplified by PCR with the following primers (each containing Illumina flow cell primers) shown in FIG. 3.

In this case, the reverse primer contains an 8 nucleotide degenerate sequence (NNNNNNNN; SEQ ID NO: XX) that acts as a molecular barcode. As described above, this barcode helps with eliminating PCR bias and can enable multiplexing of experiments.

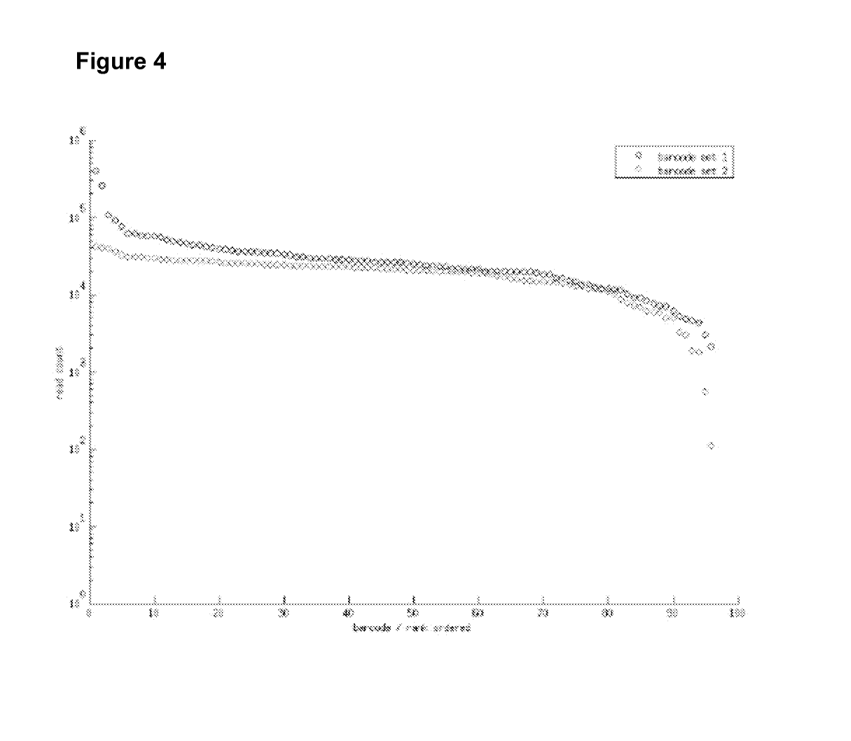

The resulting PCR amplicons were loaded on an Illumina MiSeq sequencer for a single-end 52 bp sequencing. The particle barcodes were counted and rank ordered as a function of abundance (see FIG. 4).

This illustrates that all 96 barcodes for each set were present and that the barcode loading, mixing, and amplification was uniform across each set (˜90/96 barcodes fall between 10̂4-10̂5 read counts for both sets). In theory, the perfect scenario would be where every barcode is present at the same frequency (e.g. a straight flat line). In practice, this is probably about as close to ideal as possible due to the stochastic nature of the process. Provided that barcoded nanoparticles are sequenced before and after the screening process, one can normalize out any barcode specific effects.

### Example 2

### Peptide Based Approaches

Similar to the strategy used for nucleic acids, but short peptides, peptoids, and specific patterns of isotope labeling are used to encode particle identity. Mass spectrometry could then be used as a read-out for particle identity and abundance (see, for example, Zhou et al., ACS Chemical Biology, 2(5):337-46 (2007)).

### Example 3

### Optical Encoding

Different combinations of fluorescent dyes, quantum dots, nanodiamonds, or FRET systems (using components with sufficient spectral separation and unique optical properties) could be embedded within or covalently attached to the surface of particles. The optical properties of the selected particles can then be read using a flow cytometer or a microscope for identification (see, for example, Han et al., Nat Biotech. 19:631-35 (2001); Xu & Bakker, Anal Chem. 79:3716-23 (2007); Pregibon et al., Science 315:1393-96 (2007)).

### Example 4

### Mass-Encoded Particles

Lanthanide atoms, either single species or combinations thereof, could be embedded within or attached to the surface of particles. Mass spectrometry can then be used as a read-out for particle identity. For example, particles labeled with stable heavy metal isotopes using time-of-flight atomic mass cytometry technology (e.g., FLUIDIGM® CyTOF® 2 mass cytometer).

### Example 5

### Combining Single Cell Profiling with Tagged, Functionalized Particles

Solid cancer tumors are comprised of genetically and phenotypically heterogeneous populations of cells due to a high rate of somatic mutation, clonal expansion, and diverse tumor microenvironment. This genetic and/or phenotypic diversity can also manifest itself as cells are released from a primary tumor and begin to transit the circulatory system as circulating tumor cells (CTCs).

A tumor or a collection of CTCs can be exposed to a collection of functionalized particles to characterize, at the single cell level, the identity of the specific functionalized particles linked to a functional moiety or combination of moieties that are bound to a cell of a particular genotype and phenotype.

Tissues or cells can be contacted with a collection of functionalized particles as described herein, sorted into physically separated wells then the cell genotype and/or phenotype is characterized (for example, through whole genome sequencing, RNAseq, exome capture, mass spectrometry, etc.), and the unique tags linked to the functionalized particles can be used to determine the functional moiety or combination of moieties associated with the particular genotype or phenotype. Cell sorting can be achieved using a limiting dilution approach, a microfluidic single cell isolation platform like the Fluidigm Cl, a bulk cell sorter (BD FACSAria), or a combination of these approaches. Following library preparation for both the functionalized particles' tag and the cellular RNA or genomic DNA, with appropriate well-specific barcodes, multiple samples can be pooled together for a single sequencing run. One advantage of this approach is that the cells for analysis can be pre-selected (i.e., gated if cell sorting is used) based on a biomarker of interest (assuming the marker can be fluorescently labeled). This approach is potentially limited by throughput, as one needs a unique well tag for each additional cell being interrogated in a single experiment. This can potentially be alleviated with advances in massively parallel DNA synthesis.

Instead of sorting the sample of cells and functionalized particles into physically separated wells, one could perform in situ sequencing to identify both the functionalized particle tag and basic genotype/phenotype information for cells in a massively parallel approach. Cells coated with functionalized particles can be embedded within a gel matrix and physically separated on a glass slide, for example, and the RNA can be sequenced directly in place within the cell (for example, see Lee et al., “Highly multiplexed subcellular RNA sequencing in situ” Science 343:1360-63 (2014); Lee et al., Nature Protocols 10(3):442-58 (2015)), for an example of in situ sequencing for obtaining 30 bp reads from >8000 different genes in single cells). Using this approach, in conjunction with in situ sequencing of barcodes on the functionalized particles (e.g. see Gu et al., “Multiplexed single molecule interaction profiling of DNA barcoded proteins” Nature 2014), would enable single cell resolution of what specific cellular phenotypes are bound by specific single functionalized particle types. The drawback to this approach is the relatively short reads currently accessible (currently on the order of 30 bp) and the subset of the total genome/transcriptome accessible for in situ sequencing.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A tagged, functionalized particle comprising one or more functional moieties and a unique tag linked to a particle.

2. The functionalized particle of claim 1, wherein the unique tag is a nucleic acid, a peptide, an optically-encoded tag or a mass-encoded tag.

3. The functionalized particle of claim 2, wherein the unique tag is nucleic acid and the nucleic acid is DNA, RNA or XNA.

4. The functionalized particle of claim 3, wherein the nucleic acid is comprised of 5-150 nucleotides of known sequence.

5. The functionalized particle of claim 4, wherein the nucleic acid is single stranded.

6. The functionalized particle of claim 4, wherein the nucleic acid is double stranded.

7. The functionalized particle of claim 2, wherein the peptide is a short peptide, a peptoid, or a specific pattern of isotope labeled peptide.

8. The functionalized particle of claim 7, wherein the peptide or peptoid is 5-20 amino acids of known sequence.

9. The functionalized particle of claim 2, wherein the optically-encoded tag is one or a combination of fluorescent dyes, quantum dots, polymer dots, nanodiamonds, or FRET systems.

10. The functionalized particle of claim 2, wherein the mass-encoded tag is a lanthanide atom, either single species or combinations thereof, and is embedded within or attached to the surface of the particle.

11. The functionalized particle of claim 1, wherein at least one functional moiety is a monoclonal antibody, a polyclonal antibody, single chain antibody, a single domain antibody, a bi-specific antibody, an affibody molecule, a peptide, a peptoid, an aptamer, a small molecule or a chemical compound.

12. The functionalized particle of claim 1, wherein the functionalized particle comprises a first and a second functional moieties and the second functional moiety is different than the first functional moiety and is a monoclonal antibody, a polyclonal antibody, single chain antibody, a bi-specific antibody, a small molecule or a chemical compound.

13. The functionalized particle of claim 1, wherein the particle is a bead.

14. A method of preparing a tagged, functionalized particle comprising linking one or more functional moieties and a unique tag to a particle.

15. The method of claim 14, wherein the unique tag is a nucleic acid, a peptide, an optically-encoded tag or a mass-encoded tag.

16. The method of claim 15, wherein the nucleic acid is DNA, RNA or XNA.

17. The method of claim 15, wherein the unique tag is a nucleic acid and the nucleic acid is comprised of 5-150 nucleotides of known sequence.

18. The functionalized particle of claim 17, wherein the nucleic acid is single or double stranded.

19. The functionalized particle of claim 17, wherein the nucleic acid is single or double stranded.

20. The method of claim 15, wherein the peptide is a short peptide, a peptoid, or a specific pattern of isotope labeled peptide.

21. The method of claim 20, wherein the peptide or peptoid is 5-20 amino acids of known sequence.

22. The method of claim 15, wherein the optically-encoded tag is one or a combination of fluorescent dyes, quantum dots, polymer dots, nanodiamonds, or FRET systems (using components with sufficient spectral separation and unique optical properties).

23. The method of claim 15, wherein the mass-encoded tag is a lanthanide atom, either single species or combinations thereof, and is embedded within or attached to the surface of the particle.

24. The method of claim 14, wherein at least one functional moiety is a monoclonal antibody, a polyclonal antibody, a single chain antibody, a single domain antibody, a bi-specific antibody, an affibody molecule, a peptide, a peptoid, a small molecule, an aptamer or a chemical compound.

25. The method of claim 14, wherein the functionalized particle comprises a first and a second functional moieties and the second functional moiety is different than the first functional moiety and is a monoclonal antibody, a polyclonal antibody, single chain antibody, a bi-specific antibody, a small molecule or a chemical compound.

26. The method of claim 14, wherein the particle is a bead.

27. A method of screening a collection of uniquely tagged, functionalized particles to identify a functional moiety or combination of functional moieties having a desired property or properties, the method comprising preparing a collection of tagged, functionalized particles of claim 1, introducing the collection of tagged, functionalized particles into an assay to select for a specific property or properties; isolating the tagged, functionalized particles that manifest the desired property or properties; identifying the tags of the isolated tagged functionalized particles; and determining the functional moiety or combination of functional moieties from the identity of the tag.

28. The method of claim 27, wherein the assay is in vitro or in vivo.

29. The method of claim 27, wherein the desired property is a location, a concentration, a binding affinity, a pharmacokinetic, a pharmacodynamic or a chemical property of the functionalized particle.

30. The method of claim 27, wherein the tags are identified by nucleotide sequencing if the tags are nucleic acids; mass spectrometry if the tags are peptides or mass-encoded; or flow cytometry or microscopy if the tags are fluorescent dyes, quantum dots or nanodiamonds.


SEQUENCE LISTING (7 sequences)

SEQ ID NO: 1 - DNA, 58 bp, Artificial Sequence
  Synthetic oligonucleotide
aatgatacggcgaccaccgagatctacactctttccctacacgacgctcttccgatct

SEQ ID NO: 2 - DNA, 22 bp, Artificial Sequence
  Synthetic oligonucleotide
aatgatacggcgaccaccgaga

SEQ ID NO: 3 - DNA, 68 bp, Artificial Sequence
  Synthetic oligonucleotide
caagcagaagacggcatacgagatnnnnnnnnnngtgactggagttcagacgtgtgctcttccgatct

SEQ ID NO: 4 - DNA, 24 bp, Artificial Sequence
  Synthetic oligonucleotide
caagcagaagacggcatacgagat

SEQ ID NO: 5 - DNA, 52 bp, Artificial Sequence
  Synthetic oligonucleotide
acacgacgctcttccgatctnnnnnnnnagatcggaagagcacacgtctgaa

SEQ ID NO: 6 - DNA, 8 bp, Artificial Sequence
  Synthetic oligonucleotide
nnnnnnnn

SEQ ID NO: 7 - DNA, 68 bp, Artificial Sequence
  Synthetic oligonucleotide
agatcggaagagcacacgtctgaactccagtcacnnnnnnnnnnatctcgtatgccgtcttctgcttg